(19) United States
(12) Patent Application Publication
(10) Pub. No.: US 2010/0015216 A1
(43) Pub. Date: Jan. 21, 2010

(54) METHODS AND MATERIALS FOR TREATING ACNE

(76) Inventor: GEORGIA LEVIS, New York, NY (US)

Correspondence Address: GIFFORD, KRASS, SPRINKLE,ANDERSON & CITKOWSKI, P.C, PO BOX 7021, TROY, MI, 48007-7021, US

(21) Appl. No.: 12/504,773

(22) Filed: Jul. 17, 2009

Related U.S. Application Data
(60) Provisional application No. 61/081,417, filed on Jul. 17, 2008.

(51) Int. Cl. A61K 9/127; A61K 31/60; A61K 31/185

(52) U.S. Cl. 424/450; 514/159; 514/578

(57) ABSTRACT

Compositions and methods for the treatment of acne are based upon a first therapeutic agent which is one or more members selected from the group consisting of: salicylic acid, azelaic acid, adapalene, benzoyl peroxide, and antibiotics; and a second therapeutic agent which comprises a taurine species. The two agents may be present in a single preparation and applied to the skin simultaneously, or they may be present in two separate preparations and applied to the skin sequentially. The taurine species may comprise free taurine, taurine salts, taurine esters, taurine complexes, taurine conjugates and taurine precursors either singly or in combination. The composition may include a liposomal carrier. Further disclosed are specific preparations and methods used for the treatment of acne.

### CROSS-REFERENCE TO RELATED APPLICATION

This application claims priority of U.S. Provisional Patent Application Ser. No. 61/081,417 filed Jul. 17, 2008, entitled “Methods and Materials for the Treatment of Acne,” the disclosure of which is incorporated herein by reference.

### FIELD OF THE INVENTION

This invention relates generally to topical therapies and materials for treating acne. More specifically the invention relates to compositions and methods for the treatment of acne. More specifically, the invention relates to taurine based compositions and methods used for treating acne.

### BACKGROUND OF THE INVENTION

Acne, as used herein, is to be understood to refer to a group of disorders of the skin which are characterized by redness and irritation, usually accompanied by the presence of papules and/or pustules. In the context of this disclosure “acne” refers to specific conditions which include, but are not limited to, acne vulgaris, rosacea and seborrhoeic dermatitis.

Taurine is a biomolecule which contains a sulfonate group and an amine group. As such, taurine is broadly classified as an amino acid even though it does not include a carboxylate function. Taurine is not incorporated into protein, but it is present in high concentrations in mammalian plasma and cells, and plays an important role in a number of essential biological processes such the development of the central nervous system and the retina, calcium, modulation membrane stabilization, reproduction and immunity. Furthermore, it is has been found that within the cell, taurine plays a role in osmoregulation, inhibition of apoptosis and healing processes. Taurine can promote the hydration of skin. Taurine also exerts an effect on ionic transport and can relax the skin. Furthermore, taurine promotes healing of the skin.

It is to be noted that within the context of this disclosure, the term “taurine species” is used to refer to both free amino acid as well as related materials such as its derivates, salts, esters, complexes, precursors and the like. Such derivatives and complexes specifically include taurine and taurolidine. Such derivates also include combinations or other complexes of taurine with organic acids such as glycolic acid, ascorbic acid halocarboxylic acids, and amino acids among other materials. These derivates and complexes can also include products formed by the reaction of taurine with mineral acids or bases. Also included within the definition of taurine species are precursors of taurine both biological and synthetic, and such precursors are understood to include molecules which break down into, and are metabolized to, or otherwise generate taurine in vitro. Some taurine precursors include cysteine and methionine.

The prior art has previously suggested that certain taurine derived materials can exert an antibacterial effect and, as such, may have utility in the treatment of acne. In this regard, published U.S. patent application 2004/0214891 shows the use of taurine-bromamine as a bactericide in soaps and the like which may be used for the treatment of acne. Published U.S. patent application 2005/0008684 shows methods for the treatment of skin conditions, including acne, by the use of taurolidine and/or taurultam based compositions. Despite such teaching, the prior art has not successfully developed effective therapies or materials for the treatment of acne based upon the use of various species of taurine materials. As will be disclosed herein, the present invention provides highly effective acne therapies based upon synergistic formulations of taurine species and other therapeutic materials and/or liposomal vehicles. These and other advantages of the invention will be apparent from the discussion and description which follow.

### SUMMARY OF THE INVENTION

Disclosed is a composition for the treatment of acne. The composition includes a first therapeutic agent which is selected from the group consisting of salicylic acid, azelaic acid, adapalene, benzoyl peroxide, and antibiotics taken either singly or in combination. The composition also includes a second therapeutic agent which comprises a taurine species. In some instances, the composition may comprise a single preparation including both of the therapeutic agents so that the two agents may be applied to the skin simultaneously. In other instances, the composition may comprise two separate formulations, a first including the first therapeutic agent and a second including the second therapeutic agent. In such two-part compositions, the two therapeutic agents may be applied to the skin sequentially.

The taurine species may, in some instances, comprise one or more of: free taurine, taurine salts, taurine esters, taurine complexes, taurine conjugates, and taurine precursors. In certain instances, the composition may include a liposomal carrier, and one particular group of liposomal carriers which may be used in the present invention is based upon diacylglycerol-PEG.

In other instances, the composition may further include a third therapeutic agent which may comprise one or more of: a retinoid, a hydroxyacid such as glycolic acid, ascorbic acid, vitamin A, urea, and antibiotics. The composition may also include a material which upregulates the activity of the taurine channel receptor in the skin and/or activates the taurine transport channel. Other ingredients in the composition may include permeation enhancers such as glycols and surfactants.

In specific instances, the first therapeutic agent is present in the range of 0.01%-50%, on a weight basis. In certain instances, the taurine species is present in tie range of0.01-10% on a weight basis.

Further disclosed are methods for treating acne based upon the use of these compositions.

### DETAILED DESCRIPTION OF THE INVENTION

The present invention recognizes that taurine has a number of beneficial effects on the skin which make it useful in the treatment of acne. In this regard, taurine species have been shown to have an anti-microbial effect. Taurine species also have demonstrated anti-oxidant effects, anti-inflammatory properties, and have been shown to promote healing and moisturizing of the skin. In all of these regards, taurine species, taken alone, will exert a beneficial effect on acne conditions. In addition, the properties of taurine species also make them useful in combination with other therapeutic agents used for acne conditions. In this regard, taurine species can ameliorate side effects, such as irritation, erythema, scaling, and drying, associated with topical therapeutic agents. Also, taurine species can act to enhance the permeation and/or uptake of conventional therapeutic agents thereby enhancing their bioactivity and bioavailability.

In accord with the present invention, it has been found that taurine species have significant utility in acne formulations and therapies since these materials, in addition to having a direct therapeutic effect, interact synergistically with other therapeutic agents to reduce their side effects and/or enhance their beneficial effects.

In accordance with present invention, it has been found that compositions for the treatment of acne may be based upon a mixture of therapeutic agents. A first component of the formulation is a taurine species, and as is to be understood in context of this disclosure, this first component may include either a single taurine species or a mixture of taurine species. The taurine species, in some particular instances, comprises a complex of taurine and a haloamine, and some particular haloamines comprise chloramines or bromines.

The composition also includes one or more second therapeutic agents having a beneficial effect on acne. There are large numbers of such agents, and some particular agents include benzoyl peroxide, salicylic acid, azelaic acid, and adapalene. Other therapeutic agents may comprise retinoids, hydroxy acids such as glycolic acid or ascorbic acid, as well as vitamin A, urea and the like.

The concentration of the active materials maybe adjusted over a very wide range, depending upon particular applications. Generally, the taurine species are present, on a weight basis in the range of 0.01-10 percent, while the other therapeutic agents may be present over the range of 0.01-50 weight percent depending on the nature of the agent. For example, highly potent agents such as retinoids may be used at relatively low concentrations. while agents such as urea may be present in high concentrations.

As is known in the art, the composition will typically include a carrier. In the simplest incidences, the carrier may be aqueous based or based upon an organic solvent. In other instances, the compositions may be based upon cream or lotion formulations as are well known in the art. Such carriers may include various emulsions, vesicular structures and the like, and in particular instances, the carriers are based upon liposomes. In yet other instances, gels or foams may be used as carriers.

One specific group of liposome based carriers having utility in the present invention is a group of self-forming, thermodynamically stable liposomes based upon diacylglycerol-PEG lipids. Such liposomal materials are disclosed, for example, in U.S. Pat. Nos. 6,610,322; 6,958,160; and 7,150,883, the disclosures of which are incorporated herein by reference. It has been found that compositions which include taurine materials and these particular liposomal carriers can be highly effective in the treatment of acne, either with or without the inclusion of a second therapeutic material. This increased efficacy is believed to be a result of the inclusion of this particular liposomal carrier.

The therapeutic compositions may include other active materials which serve to enhance the penetration of the various therapeutic components into the skin. Such materials include glycols such as propylene glycol and butylene glycol, as well as surfactants and other permeation enhancers of the type well known in the art.

Transport of taurine species into the skin may also be enhanced by the use of agents which upregulate the activities of taurine channel receptor in the skin. These materials can act to activate the taurine transport channel thereby promoting the uptake of the taurine material. Among the materials having this activity are those which activate an ion channel in the skin and/or mimic at least some of the intracellular activities of ATP. Such taurine transport enhancers can include hydroxy acids such as glycolic acid, as well as ascorbic acid, vitamin A, and urea, as well as their derivatives.

Compositions of the present invention may further include other therapeutically active agents such as topical anesthetics, antibiotics, and antibacterial agents. Some antibiotics having utility in these formulations include: clindamycin, erythromycin, clarithromycin, azithromycin, neomycin, polymyxin B, bacitracin and dapsone. Further ingredients such as sunscreens, fragrances, emollients, rheology control agents and the like can also be incorporated into the composition. In view of the teaching presented herein, one of ordinary still in the art can readily prepare various compositions I accord with the present invention.

A clinical evaluation of a composition in accord with the present invention was carried out. The composition included taurine together with salicylic acid and glycolic acid and was formulated as a liposomal preparation utilizing the self-forming, thermodynamically stable, diacylglycerol-PEG lipid base technology discussed above.

Specifically, the preparations used in this evaluation comprise, on a weight/weight basis:

| | | | |
|---|---|---|---|
| 1. | Water | 120.7 g | 60.35% |
| 2. | Taurine | 6.0 g | 3.00% |
| 3. | Salicylic acid | 4.0 g | 2.00% |
| 4. | Glycolic acid | 8.0 g | 4.00% |
| 5. | Sodium hydroxide | 1.5 g | 0.75% |
| 6. | Caprylic-capric triglyceride | 4.0 g | 2.00% |
| 7. | Glyceryl stearate | 8.0 g | 4.00% |
| 8. | Cetyl alcohol | 8.0 g | 4.00% |
| 9. | Glyceryl distearate (GDS-12) | 6.0 g | 3.00% |
| 10. | Isopropyl palmitate | 5.0 g | 2.50% |
| 11. | Cyclomethicone | 3.0 g | 1.50% |
| 12. | Dimethicone | 3.0 g | 1.50% |
| 13. | Cetearyl alcohol | 4.0 g | 2.00% |
| 14. | Softsan 142 | 3.0 g | 1.50% |
| 15. | Sorbitan stearate | 7.0 g | 3.50% |
| 16. | PEG-40 Stearate | 2.0 g | 1.00% |
| 17. | PEG-100 Stearate | 8.0 g | 4.00% |
| 18. | Uniphen P-23 | 2.8 g | 1.40% |

The composition was prepared by mixing ingredients 1-5 in a beaker, heating to a temperature in the range of 65-75° C., and mixing until uniform. Ingredients 6-17 were disposed in a separate beaker, heated to a temperature in the range of 60-70° C., and mixed until uniform. The mixture was cooled to a temperature of below 40° C. Thereafter, the mixture of the first five ingredients was added to the mixture of ingredients 6-17. Ingredient 18, which comprises a preservative which is a mix of ethyl, methyl, propyl, and butyl esters of parahydroxy benzoic acid in a 2-phenoxyethanol solvent, was added and the resulting mixture stirred to produce a final, liposomal, cream preparation.

The thus-prepared preparation was used in a longitudinal controlled clinical trial for the treatment of acne. The study was carried out with 9 participants assessed at baseline and at weekly intervals. In the study, the composition of the present invention was applied to the right side of each patient's face, and a conventional, widely used, benzoyl peroxide acne preparation was applied to the left side of each patient's face. Patients were evaluated on a weekly basis and evaluation included a general assessment (GA), a count of inflamed lesions (IL) and non-inflamed lesions (NI), scaling (SC), erythema (ER), itching (IT), burning (BU), stinging (ST), and discoloration (PI). In addition, at each evaluation, the subjects were asked to state a preference for one or the other medication.

In the evaluation, paired t-tests were performed to determine whether the observed differences in the degree of change seen on the right and left sides of the face were likely to represent a real effect or could be due to chance differences among the measures. The differences in the degree of change in discoloration (PI) and scaling (SC) were statistically significant, though a statistical trend toward significance was seen in the differences in the degree of change in the general assessment and in the number of infected lesions. The right side of each patient's face, which was treated with the formulation of the present invention, was significantly improved compared to the left side, which was treated with the prior art formulation, with regard to scaling (p<0.037) and post-inflammatory hyperpigmentation (discoloration) (p<0.008). The general assessment and inflamed lesions were better (p<0.07) on the side treated with the composition of the present invention as compared to the side treated with the prior art composition.

Additional analyses were undertaken using the fill set of longitudinal general assessments to determine whether any difference between left and right sides of the patients' faces increased over time. Generalized estimating equations (GEEs) revealed a significant influence of time on the general assessments for each side of the face (left side, p<0.002; right side, p<0.001).

In addition to the clinical measurements, the subjects were asked for their assessment of the medications and their effects. All 9 of the subjects said they preferred the right side medicine of the present invention (2-tailed binomial p<0.004). This trial demonstrated that while both the prior art and present therapies were effective for improving acne as assessed by several clinical measures, scaling and discoloration were significantly improved by the compositions of the present invention. Also, it is very significant that the composition of the present invention was preferred by 9 out of 9 of the subjects.

Although the foregoing describes therapeutic materials having all of their active agents mixed into one composition, it is to be understood that therapies in accordance with the present invention may also be implemented by the sequential application of active materials and other materials to the skin. In view of the teaching presented herein, various other modes of therapy and compositions will be readily apparent to those of skill in the art. It is the following claims, including any equivalents, which define the scope of the invention.

1. A composition for the treatment of acne, said composition comprising:
a first therapeutic agent selected from the group consisting of: salicylic acid, azelaic acid, adapalene, benzoyl peroxide, antibiotics and combinations thereof; and
a second therapeutic agent which comprises a taurine species.

2. The composition of claim 1, wherein said taurine species is selected from the group consisting of: free taurine, taurine salts, taurine esters, taurine complexes, taurine conjugates, taurine precursors, and combinations thereof.

3. The composition of claim 2, wherein said taurine complex comprises a taurine/haloamine complex.

4. The composition of claim 3, wherein said taurine/haloamine complex comprises taurine/chloramine or taurine/bromamine.

5. The composition of claim 1, wherein said composition further includes a liposomal carrier.

6. The composition of claim 5, wherein said liposomal carrier includes liposomes based upon diacylglycerol-PEG.

7. The composition of claim 1, wherein said composition includes a third therapeutic agent selected from the group consisting of: a retinoid, glycolic acid, ascorbic acid, vitamin A, urea, antibiotics, and combinations thereof.

8. The composition of claim 1, further including a material which upregulates the activities of the taurine channel receptor in the skin.

9. The composition of claim 1, further including a material which activates the taurine transport channel.

10. The composition of claim 1, further including a hydroxy acid.

11. The composition of claim 1, further including a permeation enhancer.

12. The composition of claim 1, wherein the first therapeutic agent is present in the range of 0.01%-50% on a weight basis.

13. The composition of claim 1, wherein the taurine species is present in the range of 0.01%-10% on a weight basis.

14. A method for treating acne, said method comprising applying the composition of claim 1 to the skin.

15. A method for the treatment of acne, said method comprising applying to the skin:
a first therapeutic agent selected from the group consisting of: salicylic acid, azelaic acid, adapalene, benzoyl peroxide, antibiotics and combinations thereof; and
a second therapeutic agent which comprises a taurine species.

16. The method of claim 15, further including: applying to the skin, a third therapeutic agent selected from the group consisting of: a retinoid, glycolic acid, ascorbic acid, vitamin A, urea, antibiotics, and combinations thereof.

17. The method of claim 15, further including: applying to the skin, a material which upregulates the activities of the taurine channel receptor in the skin.

18. The method of claim 15, wherein the first therapeutic agent and the second therapeutic agent are applied to the skin simultaneously.

19. The method of claim 15, wherein the first therapeutic agent and the second therapeutic agent are applied to the skin sequentially.

20. A composition for the treatment of acne, said composition comprising:
a therapeutic agent which comprises a taurine species; and
a liposomal carrier which carrier includes liposomes based upon diacylglycerol-PEG.
